(19) United States
(12) Patent Application Publication
(10) Pub. No.: US 2025/0090731 A1
(43) Pub. Date: Mar. 20, 2025

(54) BIODEGRADABLE IRON-CONTAINING COMPOSITIONS, METHODS OF PREPARING AND APPLICATIONS THEREFOR

(71) Applicant: UNIVERSITY OF PITTSBURGH - OF THE COMMONWEALTH SYSTEM OF HIGHER EDUCATION, PITTSBURGH, PA (US)

(72) Inventors: PRASHANT N. KUMTA, PITTSBURGH, PA (US); SUNG JAE CHUNG, PITTSBURGH, PA (US); PARTHA SAHA, PITTSBURGH, PA (US); OLEG VELIKOKHATNYI, PITTSBURGH, PA (US); MONI KANCHAN DATTA, PITTSBURGH, PA (US); DAE HO HONG, PITTSBURGH, PA (US); DA-TREN CHOU, PITTSBURGH, PA (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 18/968,598

(22) Filed: Dec. 4, 2024

Related U.S. Application Data
(63) Continuation-in-part of application No. 18/113,676, filed on Feb. 24, 2023, now Pat. No. 12,171,912.
Application No. 18/113,676 is a division of application No. 16/559,810, filed on Sep. 4, 2019, now Pat. No. 11,590,266.
Application No. 16/559,810 is a division of application No. 14/045,011, filed on Oct. 3, 2013, now Pat. No. 11,376,349.
(60) Provisional application No. 61/710,338, filed on Oct. 5, 2012.

(51) Int. Cl. A61L 31/02; A61L 31/14; C22C 33/10; C22C 38/00; C22C 38/04; C22C 38/14; C22C 45/02

(52) U.S. Cl. CPC A61L 31/022; A61L 31/148; C22C 33/10; C22C 38/00; C22C 38/002; C22C 38/04; C22C 38/14; C22C 45/02

(57) ABSTRACT

The invention relates to biodegradable iron alloy-containing compositions for use in preparing medical devices. In addition, biodegradable crystalline and amorphous compositions of the invention exhibit properties that make them suitable for use as medical devices for implantation into a body of a patient. The compositions include elemental iron, and one or more elements selected from manganese, magnesium, zirconium, zinc and calcium. The compositions can be prepared using a high energy milling technique. The resulting compositions and the devices formed therefrom are useful in various surgical procedures, such as but not limited to orthopedic, craniofacial, tracheal, and cardiovascular.

### CROSS REFERENCE TO RELATED APPLICATIONS

This continuation-in-part application claims priority to U.S. patent application Ser. No. 18/113,676, filed Feb. 24, 2023, entitled “BIODEGRADABLE IRON-CONTAINING COMPOSITIONS, METHODS OF PREPARING AND APPLICATIONS THEREFORE”, which is a divisional application of and claims priority under 35 U.S.C. § 119 (c) to U.S. patent application Ser. No. 16/559,810, filed Sep. 4, 2019, and issue as U.S. Pat. No. 11,590,266 on Feb. 28, 2023, entitled “BIODEGRADABLE IRON-CONTAINING COMPOSITIONS, METHODS OF PREPARING AND APPLICATIONS THEREFOR”, which is a divisional application of and claims priority to U.S. patent application Ser. No. 14/045,011, filed Oct. 3, 2013, and issued as U.S. Pat. No. 11,376,349 on Jul. 5, 2022, entitled “BIODEGRADABLE IRON-CONTAINING COMPOSITIONS, METHODS OF PREPARING AND APPLICATIONS THEREFOR”, which claims priority to U.S. Provisional Patent Application Ser. No. 61/710,338, filed Oct. 5, 2012, entitled “BIODEGRADABLE IRON-CONTAINING COMPOSITIONS, METHODS OF PREPARING AND APPLICATIONS THEREFOR,” the contents of which are herein incorporated by reference.

### GOVERNMENT SUPPORT

The invention was made with government support under EEC-0812348 awarded by the National Science Foundation. The government has certain rights in the invention.

### FIELD OF THE INVENTION

The invention relates to metal alloy-containing compositions and articles, and methods of their preparation. The invention is particularly suitable for use in fabricating biodegradable materials and medical devices for implantation into a patient body, such as for example, orthopedic, craniofacial and cardiovascular implant devices.

### BACKGROUND OF THE INVENTION

Metallic implant devices, such as plates, screws, nails and pins are commonly used in the practice of orthopedic and craniofacial implant surgery, and metallic stents are also implanted into a patient body to support lumens, for example, coronary arteries. These metallic implant devices are typically constructed of stainless steel, cobalt-chrome alloys, platinum or titanium, and titanium-based alloys. An advantage of these materials of construction is that they exhibit good biomechanical properties. However, a disadvantage is that implant devices constructed of these materials do not degrade over a period of time. Thus, the patient may require a second surgery to remove the implant device when there is no longer a medical need for the device to remain in the patient. For example, in certain instances, such as pediatric applications, there may be a concern that if an implant device remains in the patient's body after it is determined that there is no longer a need for it, the device may eventually be rejected by the body and cause complications for the patient. Furthermore, there are complications related to bone plate growth misalignments such as slipped capital femoral epiphysis (SCFE) with the presence of non-degradable metallic implants remaining in the body of the pediatric patients. Thus, there is room for improvement in medical implant devices and, particularly, in the materials for construction of these devices. For example, it would be advantageous for: (i) the implant device to be constructed of material that is capable of degrading over a period of time, (ii) the implant device to dissolve in a physiological environment in a controlled manner such that it would not remain in the body when there is no longer a medical need for it, and (iii) a patient not to be subjected to surgery in order to remove the implant device from its body.

Conventional biomaterials used for load bearing orthopedic and craniofacial applications are primarily chosen based on their ability to withstand cyclic loads. Metallic biomaterials, in particular, typically exhibit properties such as high strength, ductility, fracture toughness, hardness, corrosion resistance, formability, and biocompatibility to make them attractive for most load bearing applications. The most prevalent metals known for load-bearing applications are stainless steels, titanium (Ti), and cobalt-chromium (Co—Cr) based alloys, although their stiffness, rigidity, and strength far exceed those of natural bone. Further, their elastic modulus differs significantly from natural bone causing stress-shielding effects that may lead to reduced loading of bone and this decrease in stimulation may result in insufficient new bone growth and less implant stability. With conventional metallic biomaterials there is also a potential risk of toxic metallic ions and particles being released into the patient's body at the implant site through corrosion or wear which may cause an immune response. Implant devices constructed of conventional metallic biomaterials may also lead to hypersensitivity, growth restriction (most significantly for pediatric implants), implant migration, and imaging interference. Due to these complications, it is estimated that 10% of patients having implants may require surgery to remove or replace the implants, e.g., metallic plates and screws, exposing these patients to additional risks, and increasing surgical time and resources.

There is a need and desire to design and develop new load-bearing biomaterials with the objectives of providing adequate support while the natural bone is healing and allowing the implant device to harmlessly degrade over time in the patient's body when the implant device is no longer needed to perform its function in the body.

As a result of this need, degradable biomaterials have recently been developed employing resorbable polymers. However, resorbable polymer fixation plates and screws have been shown to be relatively weaker exhibiting inferior fracture toughness, and less rigid as compared to conventional metallic biomaterials, and have demonstrated local inflammatory reactions. Biodegradable materials which are used as replacements for conventional metallic biomaterials in the construction of implant devices include polymers, such as polyhydroxy acids, polylactic acid (PLA), polyglycolic acid (PGA), and the like. These materials have been found to exhibit relatively poor strength and ductility, and moreover, they do not have any osteogenic characteristics promoting osteoconduction and more importantly, osteoinduction and additionally, have a tendency to react with human tissue which can limit bone growth.

To overcome the disadvantages associated with resorbable polymer, iron (Fe)-based alloys have emerged as new biodegradable materials for cardiovascular and orthopedic applications. Iron-based alloys are not as actively and vigorously reactive with body fluids such as the magnesium and magnesium alloy counterparts causing copious amounts of hydrogen to be released, and thus, have been found to degrade when implanted without producing harmful hydrogen gas. The evolution of hydrogen, such as, hydrogen bubbles may result in complications within a body of a patient. Further, iron-based alloys have been found to possess better mechanical properties, e.g., high strength, in comparison to the biodegradable magnesium-based alloys. For example, iron has been investigated as a biodegradable stent and showed no significant obstruction of the stented vessel. However, iron is known to degrade very slowly. Furthermore, the various known biodegradable iron-based alloys can exhibit low biocompatibility and/or low corrosion rates, which render these materials unsuitable for use as implant devices.

In the field of biomedical applications, there is a desire to develop improved biodegradable metal alloy-containing implant materials having good compressive strength, corrosion rate matching time of healing of the surrounding tissue, and biocompatibility.

### SUMMARY OF THE INVENTION

In one aspect, the invention provides a biodegradable, metal alloy-containing composition including elemental iron, and one or more elements selected from the group consisting of manganese, magnesium, zinc, zirconium and calcium.

In certain embodiments, the one or more elements are manganese, magnesium and calcium. In other embodiments, the one or more elements are magnesium, zirconium and calcium, or alternatively, magnesium, zinc and calcium, or alternatively, magnesium, manganese and zirconium.

The elemental iron may be present in an amount such that it constitutes from about 10.0 weight percent to about 95.0 weight percent or from about 10.0 weight percent to about 60.0 weight percent, based on the total weight of the composition. The manganese, magnesium and calcium may be each present in an amount such that the manganese constitutes from about 5.0 weight percent to about 75.0 weight percent or from about 5.0 weight percent to about 19.0 weight percent or from about 5.0 weight percent to about 15.0 weight percent, based on the total weight of the composition; the magnesium constitutes from greater than zero weight percent to about 35.0 weight percent or from about greater than 0 weight percent to about 20.0 weight percent or from about greater than 0 weight percent to about 10.0 weight percent, based on total weight of the composition, and the calcium constitutes from greater than zero weight percent to about 10.0 weight percent, or from about greater than 0 weight percent to about 5.0 weight percent or from about greater than 0 weight percent to about 1.0 weight percent, based on the total weight of the composition. The magnesium, zirconium and calcium may be each present in an amount such that the magnesium constitutes from greater than zero weight percent to about 7.0 weight percent, the zirconium constitutes from about 8.0 weight percent to about 52.0 weight percent, and the calcium constitutes from greater than zero weight percent to about 30.0 weight percent, based on total weight of the composition. The magnesium, zinc and calcium may be each present in an amount such that the magnesium constitutes from greater than zero weight percent to about 10.0 weight percent, the zinc constitutes from greater than zero weight percent to about 10.0 weight percent, and the calcium constitutes from greater than zero weight percent to about 30.0 weight percent, based on total weight of the composition. The manganese, magnesium, and zirconium may be each present in an amount such that the manganese constitutes from about 5.0 weight percent to about 75.0 weight percent, or from about 5.0 weight percent to about 19.5 weight percent or from about 5.0 weight percent to about 15.0 weight percent or from about 5.0 weight percent to about 10.0 weight percent or from greater than 0 to about 5 weight percent, based on total weight of the composition; the magnesium constitutes from greater than zero weight percent to about 35.0 weight percent or from greater than 0 weight percent to about 20.0 weight percent or from greater than 0 weight percent to about 10.0 weight percent, based on the total weight of the composition, and the zirconium constitutes from greater than zero weight percent to about 15.0 weight percent, or from greater than zero weight percent to about 10.0 weight percent or from greater than zero weight percent to about 5.0 weight percent, or from greater than zero weight percent to about 0.5 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 19.0 weight percent, and the calcium constitutes from greater than zero weight percent to about 1.0 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 15.0 weight percent, and the calcium constitutes from greater than zero weight percent to about 5.0 weight percent, based on the total weight of the composition.

In certain embodiments, the iron constitutes from about 10.0 weight percent to about 60.0 weight percent, the magnesium constitutes from greater than zero weight percent to about 35.0 weight percent, the manganese constitutes from about 5.0 weight percent to about 15.0 weight percent, and the calcium constitutes from greater than zero weight percent to about 5.0 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 19.5 weight percent, and the zirconium constitutes from greater than zero weight percent to about 0.5 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 15.0 weight percent, and the zirconium constitutes from greater than zero weight percent to about 5.0 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 10.0 weight percent, and the zirconium constitutes from greater than zero weight percent to about 10.0 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from greater than zero to about 5.0 weight percent, and the zirconium constitutes from greater than zero to about 15.0 weight percent, based on the total weight of the composition.

In another aspect, the invention provides a method of preparing a biodegradable, metal alloy-containing composition. The method includes charging in a high energy mechanical mill elemental iron and one or more elements selected from the group consisting of manganese, magnesium, zirconium, zinc and calcium; and conducting high energy milling of the elemental iron and one or more elements.

The high energy mechanical milling process may be conducted in dry conditions followed by high energy mechanical milling conducted in wet conditions. The material resulting from the high energy mechanical milling may be subjected to a melting and casting process to form an iron alloy-containing cast alloy. The iron alloy-containing cast alloy may be finished to produce a biomedical device. The biomedical device may then be implanted into a body of a patient. The biomedical device upon implantation may then dissolve in the body of the patient without eluting copious amounts of hydrogen as desired.

In yet another aspect, the invention provides a biodegradable medical device including the aforementioned biodegradable, metal alloy-containing compositions including elemental iron, and one or more elements selected from the group consisting of manganese, magnesium, zinc, zirconium and calcium. As described above, in certain embodiments, the one or more elements are manganese, magnesium and calcium. In other embodiments, the one or more elements are magnesium, zirconium and calcium, or alternatively, magnesium, zinc and calcium, or alternatively, magnesium, manganese and zirconium.

In certain embodiments, the device is selected from the group consisting of plate, mesh, staple, screw, pin, tack, rod, suture anchor, tubular mesh, coil, x-ray marker, catheter, pipe, shield, bolt, clip, plug, dental and craniofacial implant, graft device, endoprosthesis, bone-fracture healing device, bone void filler device, bone replacement device, joint replacement device, tissue regeneration device, cardiovascular stent, tracheal stent, nerve guide, surgical implant, surgical wire, Kirschner wire, cerclage wire, and combinations thereof. The device may be an implantable device, and in certain embodiments selected from the group consisting of an orthopedic device, a craniofacial device, tracheal device, and a cardiovascular device.

### DETAILED DESCRIPTION OF THE PREFERRED EMBODIMENTS

This invention relates to novel, biodegradable metal alloy-containing compositions. Further, this invention relates to articles, such as medical devices, which are constructed or fabricated from the biodegradable metal alloy-containing compositions of this invention. Moreover, this invention relates to methods of preparing these biodegradable, metal alloy-containing compositions and articles for use in medical applications, such as but not limited to, orthopedic and craniofacial surgery.

The biodegradable metal alloy-containing compositions include iron (Fe). These Fe-containing compositions also include one or more of the following elements: zirconium (Zr), manganese (Mn), calcium (Ca), magnesium (Mg), and zinc (Zn).

The compositions of the invention can be used as materials of construction to prepare various articles, such as biomedical devices for implantation into a body of a patient for orthopedic, cardiovascular and craniofacial applications.

In addition to the desirable biodegradability property of the metal alloy-containing compositions of the invention, these compositions include at least one of the following characteristics: biocompatibility, corrosion resistance, cell attachment, cell viability and mechanical strength, which make them suitable for use as implant devices in a body of a patient.

The biodegradable metal alloy-containing compositions of the invention include the presence of iron and other elements or compounds in various amounts. In certain embodiments, the other elements can include one or more of zinc (Zn), zirconium (Zr), calcium (Ca), manganese (Mn) and magnesium (Mg). The amount of each of these elements in the compositions can however, vary. As previously indicated, in general, the amounts of each of these components are selected such that the resulting compositions exhibit one or more of the desired characteristics identified herein, e.g., acceptable non-toxic limits and desired degradability over an acceptable period of time without releasing copious amounts of unwanted hydrogen causing gas pockets upon implantation. For example, the amount of iron is selected such that the compositions exhibit corrosion resistance in the presence of water and simulated biological fluids which allow the compositions to be suitable for in vivo use, for example, in a physiological environment of pH, temperature and pressure, such as a body of a patient.

The compositions of the invention are designed to be capable of controlling the corrosion rate and improving the mechanical properties of the iron-containing alloys through the introduction of alloying elements and processing conditions. In certain embodiments, the corrosion rate matches or corresponds to the time of healing of the surrounding tissue. For example, an implantation device fabricated of the biodegradable metal alloy-containing the compositions of the invention degrades or dissolves completely at or near the time it takes for the tissue surrounding the device to heal and form the endogenous desired functional hard and soft tissue. Thus, the implantation device is not present for a prolonged period of time, e.g., a time beyond which there is a need for the device. Without intending to be bound by any particular theory, it is believed that corrosion and mechanical properties are strongly affected by the alloying elements with iron in a solid solution.

In certain embodiments, the compositions in accordance with the invention include a mixture of one or more elements, such as, Fe, Mn, Mg and Ca. In other embodiments, the compositions include a mixture Fe, Zr, Mg and Ca. In other embodiments, the compositions include Fe, Zn, Mg and Ca. In still other embodiments, the compositions include Fe, Mn, Mg and Zr. The amount of each of these elements employed can vary and in general, the amount of each of these alloying elements is selected such that the resulting compositions are within the acceptable non-toxic limits such that the compositions are sufficiently biocompatible for implantation into a body of a patient, and are fully biodegradable over a period of time so that the implantation device does not remain in the body of the patient for prolonged periods of time, e.g., beyond the period of time when there is a medical need for the implantation device.

The elemental iron may be present in an amount such that it constitutes from about 10.0 weight percent to about 95.0 weight percent or from about 10.0 weight percent to about 60.0 weight percent, based on the total weight of the composition. The manganese, magnesium and calcium may be each present in an amount such that the manganese constitutes from about 5.0 weight percent to about 75.0 weight percent or from about 5.0 weight percent to about 19.0 weight percent or from about 5.0 weight percent to about 15.0 weight percent, based on the total weight of the composition, the magnesium constitutes from greater than zero weight percent to about 35.0 weight percent or from greater than 0 weight percent to about 20.0 weight percent or from about greater than 0 weight percent to about 10.0 weight percent, based on total weight of the composition, and the calcium constitutes from greater than zero weight percent to about 10.0 weight percent, or from greater than 0 weight percent to about 5.0 weight percent or from greater than 0 weight percent to about 1.0 weight percent, based on the total weight of the composition. The magnesium, zirconium and calcium may be each present in an amount such that the magnesium constitutes from greater than zero weight percent to about 7.0 weight percent, the zirconium constitutes from about 8.0 weight percent to about 52.0 weight percent, and the calcium constitutes from greater than zero weight percent to about 30.0 weight percent, based on total weight of the composition. The magnesium, zinc and calcium may be each present in an amount such that the magnesium constitutes from greater than zero weight percent to about 10.0 weight percent, the zinc constitutes from greater than zero weight percent to about 10.0 weight percent, and the calcium constitutes from greater than zero weight percent to about 30.0 weight percent, based on total weight of the composition. The manganese, magnesium, and zirconium may be each present in an amount such that the manganese constitutes from about 5.0 weight percent to about 75.0 weight percent, or from about 5.0 weight percent to about 19.5 weight percent or from about 5.0 weight percent to about 15.0 weight percent or from about 5.0 weight percent to about 10.0 weight percent or from greater than 0 to about 5 weight percent, based on the total weight of the composition; the magnesium constitutes from greater than zero weight percent to about 35.0 weight percent or from greater than 0 weight percent to about 20.0 weight percent or from greater than 0 weight percent to about 10.0 weight percent, based on the total weight of the composition, and the zirconium constitutes from greater than zero weight percent to about 15.0 weight percent, or from greater than zero weight percent to about 10.0 weight percent or from greater than zero weight percent to about 5.0 weight percent, or from greater than zero weight percent to about 0.5 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 19.0 weight percent, and the calcium constitutes from greater than zero weight percent to about 1.0 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 15.0 weight percent, and the calcium constitutes from greater than zero weight percent to about 5.0 weight percent, based on the total weight of the composition.

In certain embodiments, the iron constitutes from about 10.0 weight percent to about 60.0 weight percent, the magnesium constitutes from greater than zero weight percent to about 35.0 weight percent, the manganese constitutes from about 5.0 weight percent to about 15.0 weight percent, and the calcium constitutes from greater than zero weight percent to about 5.0 weight percent, based on total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 19.5 weight percent, and the zirconium constitutes from greater than zero weight percent to about 0.5 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 15.0 weight percent, and the zirconium constitutes from greater than zero weight percent to about 5.0 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from about 5.0 weight percent to about 10.0 weight percent, and the zirconium constitutes from greater than zero weight percent to about 10.0 weight percent, based on the total weight of the composition.

In certain embodiments, the iron constitutes from about 10.0 weight percent to about 60.0 weight percent, the magnesium constitutes from greater than zero weight percent to about 20.0 weight percent, the manganese constitutes from greater than zero to about 5.0 weight percent, and the zirconium constitutes from greater than zero to about 15.0 weight percent, based on the total weight of the composition.

An implantation device fabricated in accordance with the invention will degrade and preferably dissolve completely within an acceptable time frame. For example, an implant device fabricated of a composition in accordance with the invention can serve as filler or support material during a natural bone and soft tissue healing process and following completion of this process, the implant device will degrade within an acceptable time period and therefore, will not remain in the body for a prolonged period of time. Suitable non-toxic limits and an acceptable time frame for degradation can vary and may depend on the physical and physiological characteristics of the patient, the in vivo site of the implantation device, and the medical use of the implantation device. Without intending to be bound by any particular theory, it is believed that the presence of iron contributes to the improved mechanical strength, ductility, fracture toughness, and controlled corrosion of the biodegradable compositions.

Non-limiting examples of medical devices in which the compositions and articles of the invention can be used include, but are not limited to, plates, meshes, staples, screws, pins, tacks, rods, suture anchors, tubular mesh, coils, X-ray markers, catheters, endoprostheses, pipes, shields, bolts, clips or plugs, dental and craniofacial implants or devices, graft devices, bone-fracture healing devices, bone void fillers, bone replacement devices, joint replacement devices, tissue regeneration devices, cardiovascular stents, tracheal stents, nerve guides, surgical implants, Kirschner wires (K-wires), cerclage, and wires. In a preferred embodiment, the medical devices include fixation bone plates and screws, temporomandibular joints, cardiovascular stents, tracheal stents, and nerve guides.

In certain embodiments, the medical implant devices described herein can have at least one active substance attached thereto. The active substance can be attached to the surface of the device or encapsulated within the device. As used herein, the term “active substance” refers to a molecule, compound, complex, adduct and/or composite that exhibits one or more beneficial activities such as therapeutic activity, diagnostic activity, biocompatibility, corrosion-resistance, and the like. Active substances that exhibit a therapeutic activity can include bioactive agents, pharmaceutically active agents, drugs and the like. Non-limiting examples of bioactive agents that can be incorporated in the compositions, articles and devices of the invention include, but are not limited to, bone growth promoting agents such as growth factors, drugs, proteins, antibiotics, antibodies, ligands, DNA, RNA, peptides, enzymes, vitamins, cells and the like, and combinations thereof.

It is contemplated that additional components may be added to the biodegradable, metal alloy-containing compositions of the invention provided that the non-toxicity and biodegradability of the compositions is maintained within the allowed and desirable acceptable limits. The additional components can be selected from a wide variety known in the art and can include but are not limited to strontium, silver and mixtures thereof.

In certain embodiments, the compositions of the invention do not include zinc. In certain other embodiments, the compositions of the invention include the presence of zinc in amounts that maintain the toxicity levels of the compositions within acceptable limits. It is known generally in the art that the presence of zinc in particular amounts, i.e., an unacceptable level, can produce an undesirable or unacceptable level of toxicity in a physiological environment, such as a body of a patient.

The biodegradable, metal alloy-containing compositions of the invention can be prepared using various methods and processes common to metallic alloy processing. In general, powder metallurgy methods and processes are employed.

For example, melting and casting processes may be employed. It is known in the art of metallurgy that melting, solidification, and casting is a production technique in which a metal or a mixture of metals is heated until it is molten and then, it is poured into a mold, allowed to cool and solidify. In one embodiment, the iron and other selected elements are melted by heating at an elevated temperature, preferably under a protective atmosphere, and then poured into a mold, allowed to cool and solidify.

Casting of the composition can be affected by using any casting procedure known in the art, such as, but not limited to, sand casting, gravity casting, direct chill casting, centrifugal casting, die casting, plaster casting and investment casting. It is believed that the particular process used for casting can affect the final physical, chemical, biological, and mechanical properties and characteristics of the cast composition. Further, it is believed that the temperature at which the melting procedure is performed can also affect the final alloy composition. Thus, the temperature may be carefully selected so as to maintain the desired composition of the alloy minimizing and preventing any volatilization losses of the composition elements.

In one embodiment, prior to solidification, the molten mixture is tested to determine the amount of the various components therein and therefore, to provide an opportunity to adjust the amounts as desired prior to solidification.

In another embodiment, the melting and/or casting steps are/is performed under a protective atmosphere to preclude, minimize or reduce the components of the composition from decomposing/oxidation. In particular, it is desired to preclude, minimize or reduce the decomposition/oxidation of magnesium in the composition. The protective atmosphere can include compounds selected from those known in the art, such as but not limited to, argon, sulfur hexafluoride and mixtures thereof.

In yet another embodiment, subsequent to the casting process, the iron alloy-containing cast alloy is subjected to homogenization. Without intending to be bound by any particular theory, it is believed that a homogenization treatment can cause the dissolution of impurities and inter-metallic phases causing complete reaction of the alloying elements to form a homogeneous solid solution.

In further embodiments, the obtained cast alloy can be subjected to various forming and finishing processes known in the art. Non-limiting examples of such processes include, but are not limited to, extrusion, forging, rolling, polishing (by mechanical and/or chemical means), surface treating (to form a superficial layer on the surface) and combinations thereof.

The resulting cast alloy can be formed, finished, machined and manipulated to produce articles and devices for use in medical applications, such as medical devices for implantation into a body of a patient. Furthermore, these medical devices can be used in orthopedic, craniofacial, tracheal, and cardiovascular applications.

In certain embodiments of the invention, Fe and one or more of Mn, Mg, Ca, Zr and Zn are alloyed by employing high energy mechanical alloying (HEMA) and uniaxial or isostatic compaction and sintering. The compositions used for HEMA can include, but are not limited to, the following: (i) Fe, Mn, Mg and Ca or (ii) Fe, Zr, Mg and Ca or (iii) Fe, Zn, Mg and Ca or (iv) Fe, Mg, Mn and Zr. For example, the Fe and other selected elements in powder form are charged to a high energy mechanical mill. Further, stainless steel (SS) balls are included in the charge with the elements. The SS balls typically used in a HEMA process have a diameter in the range of from 5 mm to 8 mm. The amount of each of the charge components can vary. In alternate embodiments, the charge ratio of the balls to the elements (e.g., powder) can be 20:1 or 10:1 or 8:1 or 5:1. Different charge ratios can cause variations in the kinetics of the milling resulting in altering the diffusion reaction of the various alloying elements. Further, the duration of the milling also can vary depending on the amount of time needed to control the diffusion of the alloying elements allowing sufficient time for complete alloying thereby producing a homogeneous solid solution alloy. In certain embodiments, the milling can be conducted for up to 15 or 20 hours. Upon completion of the milling process, a homogeneous solid solution alloy is formed.

The milling can be conducted in dry or wet conditions. In wet conditions, suitable inert solvents can be selected from the wide variety known in the art, such as, for example, but not limited to, toluene, xylene, N-methyl-2-pyrrolidone (NMP), acetonitrile and mixtures thereof. In certain embodiments, the elements are milled in dry conditions followed by milling in wet conditions.

The HEMA may be performed under a protective atmosphere to preclude, minimize, or reduce unwanted reaction or oxidation and decomposition of the elements in the compositions. In particular, it is desirable to preclude, minimize, or reduce the active reaction with oxygen causing decomposition of magnesium in the compositions. Magnesium is known to be a non-toxic metal element that degrades in a physiological environment and therefore, is considered a suitable element for use in constructing biodegradable implant devices. However, disadvantageously, the degradation of magnesium in a physiological environment yields magnesium hydroxide and hydrogen gas. This process is known in the art as magnesium corrosion. The hydrogen gas produced in the body of the patient as a result of magnesium corrosion can produce complications because the ability of the human body to absorb or release hydrogen gas is limited.

As above-mentioned, the protective atmosphere can include compounds selected from those known in the art, such as but not limited to, argon, sulfur hexafluoride and mixtures thereof.

In certain embodiments, subsequent to HEMA, amorphous metal films are synthesized by pulsed laser deposition (PLD).

Detailed exemplary procedures for performing the melting and casting processes are depicted in the examples herein.

Additional objects, advantages and features of the invention may become apparent to one of ordinary skill in the art based on the following examples, which are provided for illustrative purposes only and are not intended to be limiting in any of the compositions or their final applications for any of the intended use as biomedical and biodegradable implant devices stated above.

### EXAMPLES

### Example 1

### Fe—Mn System by HEMA, Compaction and Sintering

### Experimental Method

### 1.1 Synthesis of Sintering the Compacted Fe—Mn Based Crystalline Powder by High Energy Mechanical Alloying (HEMA)

All alloys were produced by high energy mechanical alloying (HEMA) and compaction. Elemental powders of pure elemental Fe, Mn, Mg, and Ca were commercially obtained and loaded into stainless steel vials containing 5 mm diameter stainless steel balls inside an argon-filled glove box in which the oxygen concentration was kept below 1.0 ppm. The weight of the starting mixture was approximately 60g and the total ball weight was approximately 600g (ball to powder ratio was 10:1). The mixture was subjected to dry milling in a planetary Fritsch P5 high energy Shaker Mill for up to 20 hours with 30-minute resting intervals after every one hour of milling. Post dry milling, 15 ml of toluene (anhydrous, 99.8%, Sigma-Aldrich) was loaded in the vial and the mixture was subsequently wet milled for a period of up to 8 hours to reduce the adhesion of powders on the balls and the inner surface of the milling vial. The post ball milled powders were dried and then compacted at a pressure of 2500 psi and 60 ksi using a Carver Press 4350 and Flow Autoclave System cold isostatic compaction press to produce 10 and 25.0 mm diameter discs to be sintered at a temperature of 1200° C. for 3 hours.

### 1.2 MTT Cell Viability Assay

MTT (3-(4,5-Dimethylthiazol-2-yl)-2,5-diphenyltetrazolium bromide) assay was used to assess the cytotoxicity of degradation product after 72 h immersion of specimens in culture medium. Dulbecco's modified eagle medium (α-MEM) with 10% fetal bovine serum (FBS) was used. Extract media diluted to 50%, 25%, and 10%, as well as 100% extract media were added to the 24 h cultured MC3T3 cell and, MTT assay was performed after 24 h and 72 h culture. Before adding MTT formazan salt to wells, the extract medium was replaced with a regular cell culture medium.

### 1.3 Electrochemical Corrosion Measurement

A three-electrode setup (Ag/AgCl reference electrode, platinum wire being counter electrode) was used to measure the electrochemical corrosion properties in Dulbecco's modified eagle medium (DMEM) supplemented with 10% fetal bovine serum (FBS) and 1% penicillin/streptomycin (P/S). Potentiodynamic polarization scans were performed after 15 min of immersion between −1 and −2 V at a scan rate of 1 mVs⁻¹ using a CH instrument potentiostat (CHI 604A) at ˜37.4° C. Corrosion current (i_{corr}) and potential (E_{corr}) were calculated from extrapolated data of the cathodic and anodic part of a tafel plot.

### 1.4 Summarized Results

### Anticipated Systems

A Fe-35Mn system is a potential composition of Fe-based degradable metallic biomaterials suitable for fabricating implant devices with a longer degradation time as compared to Mg-based devices. With the knowledge that different elements at different weight ratios can enhance the degradation, magnesium and calcium were evaluated to show that addition of these elements can serve to control the corrosion characteristics.

The substitution of 1-10% Mn for Mg or Ca exhibited an increase in the corrosion rate obtained by potentiodynamic polarization measurement. In both Fe—Mn—Mg and Fe—Mn—Ca, the corrosion potential increased towards more positive potential and the current density also increased, suggesting an increase in corrosion rate. Fe—Mn—Ca showed acceptable cell viability during 1 and 3 day MTT assay.

Fe-5-40% Mn—X and Fe-5-40% Mn—X—Y systems were processed where X and Y were trace elements to control the corrosion behavior with amounts below toxic thresholds. Potential elements for X and Y included known biocompatible elements such as Mg, Zn, Al, Y, Zr, Ti, Ta, Ca, Sr, and Ce in amounts greater than zero and not higher than 10%. These powder alloys were processed using HEMA, compaction, and sintering, or through additive manufacturing methods such as 3D printing or selective laser melting.

The results in Table 1 show the corrosion potential, current density and corrosion rate for Fe—Mn, Fe—Mn—Ca, Fe—Mn—Mg and pure Mg compositions, and as-extruded AZ31 from potentiodynamic polarization measurement.

TABLE 1

| | Corrosion | Corrosion | |
|---|---|---|---|
| | Potential | Current Density | Corrosion Rate |
| Materials | E_{corr} (V) | i_{corr}(μA cm⁻²) | (mm/year) |
| Pure Mg | −1.57 | 35.19 | 0.80 |
| AZ 31 | −1.48 | 19.20 | 0.43 |
| Fe—35Mn | −0.65 | −6.97 | 0.05 |
| Fe—30Mn—5Ca | −0.56 | −19.02 | 0.18 |
| Fe—30Mn—5Mg | −0.60 | −11.20 | 0.11 |

### Example 2

### Fe—Zr—Ca, Fe—Zr—Mg, Fe—Zr—Ca—Mg System by HEMA, PLD

### Experimental Method

### 2.1. Synthesis of Amorphous/Crystalline Powder by High Energy Mechanical Alloying (HEMA)

All alloys were produced by high energy mechanical alloying (HEMA) and compaction. Commercial elemental powders of pure Fe (99.9+%, <10 μm, Alfa Aesar), pure Zr (99.6%, −325 mesh, Alfa Acsar), pure Mg (99.8%, <325 mesh, Alfa Aesar), Zn (97.5%, 6-9 μm, Alfa Aesar) and Ca (99.5%, <16 mesh, Alfa Acsar) were chosen as the starting materials. For Fe—Zr—Ca systems, each composition was accurately weighed including from about 0.6 weight percent to about 29.8 weight percent of calcium, from about 13.9 weight percent to about 52 weight percent of zirconium, and a balance of iron, based on the total weight of the composition. An amorphous structure was formed above 60 weight percent of iron in the Fe—Zr—Ca system. The Fe—Zr—Mg system included from about 0.4 weight percent to about 6.9 weight percent of magnesium, from about 8.6 weight percent to about 27.8 weight percent of zirconium, and a balance of iron, based on the total weight of the composition. In Fe—Zr—Ca—Mg system, each composition was accurately weighed including from greater than zero to about 0.8 weight percent of magnesium, from about 0.6 weight percent to about 0.7 weight percent of calcium, from about 27.8 weight percent to about 28.1 weight percent of zirconium, and a balance of iron, based on the total weight of the composition. For Fe—Zn—X system and Fe—Zn—X—Y system, each composition was accurately weighed including from about 1 weight percent to about 10 weight percent of X and Y, from about 30 weight percent to about 50 weight percent of zinc, and a balance of iron, based on the total weight of the composition. Potential elements for X and Y elements were Ca, Mg, Y, Ti, Ta, Sr, and Ce as biocompatible elements. For Mg—Zn—Fe—Zr—Ca system, it was weighed including from about 1 weight percent to about 10 weight percent of iron, zirconium and calcium, from about 30 weight percent to about 50 weight percent of zinc, and a balance of magnesium, based on the total weight of the composition. For Mg—Zr—Ca system, it was weighed including from about 1 weight percent to about 10 weight percent of calcium, from about 30 weight percent to about 65 weight percent of zirconium, and a balance of magnesium, based on the total weight of the composition. The mixture of elemental powder was loaded into stainless steel (SS) vials containing 2 mm diameter SS balls as the milling media. The ball to powder weight ratio (BPR) was 15 to 1, and the total weight of the starting mixture was 3 g. The mixture was subjected to dry milling in a planetary SPEX 8000 high energy Shaker Mill for up to 10 hours with 30-minute resting intervals after every one hour of milling. Post dry milling, 2 ml of toluene (anhydrous, 99.8%, Sigma-Aldrich) was loaded in the vial and the mixture was subsequently wet milled for a period of up to 7 hours to reduce the adhesion of powders on the balls and the inner surface of the milling vial. Handling of the powders and loading of toluene were conducted inside an argon-filled glove box in which the oxygen concentration was kept below 1.0 ppm. The post ball milled powders were dried and then compacted at a pressure of 60 psi using Flow Autoclave System cold isostatic compaction press to produce 25.0 mm diameter discs to be used as targets for PLD (Pulsed Laser Deposition).

### 2.2. Synthesis of Amorphous/Crystalline Metal Coating Layer by Pulsed Laser Deposition (PLD)

All thin films were produced by PLD utilizing a 248 nm KrF excimer laser irradiation pulsed at 25 ns FWHM in a high vacuum chamber with a base pressure of 10⁻⁶Torr. In all depositions the spot size was approximately 1×3 mm, the fluence 8.3˜9.6 J/cm^{2,} the laser pulse frequency of 10 Hz and the deposition rate about 2.3 Å/s. The target to substrate distance was maintained constant at 58 mm, with targets rotated during deposition. Films were deposited at room temperature for a deposition time of 30 minutes on amorphous SiO₂ glass for glancing angle XRD and the cytocompatibility tests.

### 2.3. Cytocompatibility

Cell biocompatibility of the alloy system was evaluated for Fe—Zr—Ca, Fe—Zr—Mg and Fe—Zr—Ca—Mg systems. Each alloy system was deposited on amorphous glass by PLD and then cell viability tests were conducted. Murine MC3T3-E1 pre-osteoblast cells, murine NIH3T3 fibroblast cells, and human mesenchymal stem cells were utilized for cell culture studies. These three kinds of cell lines were cultured on deposited film of each alloy system on glass for 24 or 72 hours and assessed using the Live/Dead cell viability assay. Cells were observed and imaged using fluorescent microscopy.

### 2.4. Characterization Method

The microstructure and phase assemblage of thin films and milled powders were examined by glancing angle (Philips PW 1830 with Cu-Kα radiation) and conventional X-ray diffraction (PANalytical X'pert Pro with Cu-K_{α} radiation). A JEOL JEM2000FX operating at 200kV was used for conducting transmission electron microscopy (TEM) and obtaining conventional bright field images. TEM samples were obtained by directly depositing the film by PLD on silicon nitride supported window grids (Ted Pella, USA) for observation under the TEM. Additionally, samples were made by depositing the films on oxidized silicon wafer containing a photo-resist following the above method. Films were lifted off from the substrate by stripping the photo-resist and transferring onto standard TEM copper membrane grid (Ted Pella, USA) for observation under the TEM.

The results are shown in Tables 2, 3, 4 and 5.

### 3.0 Summarized Results

The cytocompatibility tests showed reasonable biocompatibility after 24- and 72-hours cell culture using the Live/Dead cell viability assay for each deposited thin films of Fe—Zr—Ca, Fe—Zr—Mg and Fe—Zr—Ca—Mg systems. These as-milled powder and deposited films have amorphous structure as confirmed by XRD and TEM. These results indicate the feasibility of generating biocompatible amorphous Fe-based alloy coatings.

TABLE 2
3.1 Fe—Zr—Ca System

| Composition | Structure |
|---|---|
| Fe₇₁Zr₂₉ | amorphous |
| Fe_{71.6}Zr_{27.8}Ca_{0.6} | amorphous |
| Fe_{72.2}Zr_{26.5}Ca_{1.3} | Close to amorphous |
| Fe_{70.9}Zr_{27.8}Ca_{1.3} | Close to amorphous |
| Fe_{72.8}Zr_{25.2}Ca₂ | Close to amorphous |
| Fe₇₄Zr_{22.7}Ca_{3.3} | Close to amorphous |
| Fe_{75.3}Zr₂₀Ca_{4.7} | Close to amorphous |
| Fe_{77.3}Zr_{15.8}Ca_{6.9} | amorphous |
| Fe_{63.7}Zr_{29.8}Ca_{6.5} | amorphous |
| Fe_{43.3}Zr₅₂Ca_{4.7} | crystalline |
| Fe_{56.3}Ca_{29.8}Zr_{13.9} | crystalline |

TABLE 3
3.2 Fe—Zr—Mg System

| Composition | Structure |
|---|---|
| Fe_{71.8}Zr_{27.8}Mg_{0.4}* | amorphous |
| Fe_{72.5}Zr_{26.7}Mg_{0.8}* | amorphous |
| Fe_{73.3}Zr_{25.5}Mg_{1.2}* | amorphous |
| Fe_{74.2}Zr_{24.2}Mg_{1.6}* | amorphous |
| Fe_{71.5}Zr_{26.9}Mg_{1.6}* | Close to amorphous |
| Fe_{72.8}Zr_{25.6}Mg_{1.6}* | Close to amorphous |
| Fe_{79.5}Zr_{16.2}Mg_{4.3}* | amorphous |
| Fe_{84.5}Zr_{8.6}Mg_{6.9}* | amorphous |

TABLE 4
3.3 Fe—Zr—Ca—Mg System

| Composition | Structure |
|---|---|
| Fe_{71.53}Zr_{27.79}Ca_{0.64}Mg_{0.04} | amorphous |
| Fe_{71.32}Zr_{27.84}Ca_{0.64}Mg_{0.2} | amorphous |
| Fe_{71.21}Zr_{27.87}Ca_{0.64}Mg_{0.28} | amorphous |
| Fe_{71.05}Zr_{27.91}Ca_{0.65}Mg_{0.39} | amorphous |
| Fe_{70.78}Zr_{27.98}Ca_{0.65}Mg_{0.59} | amorphous |
| Fe_{70.5}Zr_{28.06}Ca_{0.65}Mg_{0.79} | amorphous |

TABLE 5
3.4 Other Systems

| Composition | Structure |
|---|---|
| Fe_{50.8}Zn_{43.7}Ca_{5.5} | amorphous |
| Fe_{58.1}Zn_{36.3}Ca_{5.6} | amorphous |
| Fe_{51.9}Zn_{44.7}Mg_{3.4} | Close to amorphous |
| Mg_{30.1}Zr_{65.8}Ca_{4.1} | Crystalline |
| Mg_{37.1}Zn_{46.6}Fe₈Zr_{3.2}Ca_{5.1} | Close to amorphous |
| Fe_{53.2}Zn_{38.1}Mg_{2.9}Ca_{5.8} | Close to amorphous |

*The Mg in the Fe—Zr—Mg system above can include Mg-containing alloys.

### 4.0 Fe—Mg—Zn, Fe—Ca—Zn, Fe—Mn—Mg—Zn, and Fe—Mn—Ca—Zn Alloys Computational Study

It is generally known that pure Fe corrodes much slower in aqueous environments than Mg and its alloys. Fe-based alloys with improved dissolution kinetics, enabling them to degrade faster, would allow for the manufacture of materials having controlled degradation. Addition of an appreciable amount of suitable alloying elements with lower electrochemical potential in comparison to Mg was evaluated to increase the degradation rate to level suitable for bio-applications. It was evaluated whether galvanic corrosion between the different phases of the compound played a positive role in accelerating the much desirable biodegradation.

Fe-based ternary compositions were prepared according to the following compositions (weight %):

Fe_{100-x-y}MgₓZn_{y}, Fe_{100-x-y}CaₓZn_{y}, [Fe_{0.65}Mn_{0.35}]_{100-x-y}MgₓZn_{y}, and [Fe_{0.65}Mn_{0.35}]_{100-x-y}CaₓZn_{y},
wherein 5≤x≤65, 12≤y≤70 and x+y<100.

A thermodynamic evaluation of the hydrolytic reactions was conducted:

$$Fe - Mg - Zn + 2 {H}_{2} O \text{=>} [ Fe - Mg - Zn ] {( OH )}_{2} + {H}_{2} ↑ \qquad ( 1 )$$

$$Fe - Ca - Zn + 2 {H}_{2} O \text{=>} [ Fe - Ca - Zn ] {( OH )}_{2} + {H}_{2} ↑ \qquad ( 2 )$$

$$Fe - Mn - Mg - Zn + 2 {H}_{2} O \text{=>} [ Fe - Mg - Mn - Zn ] {( OH )}_{2} + {H}_{2} ↑ \qquad ( 3 )$$

$$Fe - Mn - Ca - Zn + 2 {H}_{2} O \text{=>} [ Fe - Mn - Ca - Zn ] {( OH )}_{2} + {H}_{2} ↑ \qquad ( 4 )$$

A comparison was made of the heat of the reaction ΔG⁰ with those calculated for hydrolysis of pure Fe and Mg:

$$Fe + 2 {H}_{2} O \text{=>} {Fe ( OH )}_{2} + {H}_{2} ↑ \qquad ( 5 )$$

$$Mg + 2 {H}_{2} O \text{=>} {Mg ( OH )}_{2} + {H}_{2} ↑ \qquad ( 6 )$$

Results were obtained from the thermodynamic calculations using a CALPHAD approach. The results showed that the more negative was the free energy change, the higher thermodynamic stimulus of the hydrolytic reaction was observed in comparison with pure Fe for which δΔG⁰=0. For pure Mg δΔG⁰=−361 KJ/mol there was indicated an increased propensity of Mg to react with water in comparison with Fe. The region between δΔG⁰=0 and −361 KJ/mol reflected alloys with intermediate stimulus for the reaction, which may be useful for designing alloys with controlled degradation since the corrosion rate may be directly dependent on the composition of the alloy.

### 5.0 Results

Fe-(4.4-36.6) wt. % Mg-(35.5-49.3) wt. % Zn and Fe-(4.3-27.7) wt. % Mg-(46.3-59.6) wt. % Zn were synthesized by high energy mechanical alloying (HEMA) using elemental blends of iron (Alfa Aesar 99.9%), zinc (Alfa Aesar >99.95%), and magnesium (Alfa Aesar 99.9%) powders which were mechanically milled in a high energy shaker mill (SPEX CeriPrep 8000M) for 10 h in a stainless steel (SS) vial using 20 SS balls of 2 mm diameter (˜20 g) with a ball to powder weight ratio of 10:1. To determine the phase formation in the mechanically milled powder, X-ray diffraction (XRD) was carried out using Philips PW1830 system employing the CuK_{α} (λ=0.15406 nm) radiation.

Powder XRD pattern of Fe_{49.4}Mg_{4.3}Zn_{46.3} and Fe_{41.9}Mg_{9.1}Zn₄₉ formed metastable solid solution of Zn (Fe Mg) hcp structures. With an increase of magnesium content (Fe_{33.4}Mg_{14.5}Zn_{52.1}, Fe_{23.7}Mg_{20.7}Zn_{55.6}) above 9 wt. %, the structure became amorphous which co-existed with metastable Zn (Fe Mg) hcp structure.

XRD pattern of Fe_{53.3}Mg_{9.3}Zn_{37.4} and Fe_{60.3}Mg_{4.4}Zn_{35.3} formed amorphous phase which co-exist with metastable Zn(Fe Mg) hcp structure. However, with increase of magnesium content (Fe_{36.3}Mg_{21.1}Zn_{42.6}, Fe_{45.4}Mg_{14.8}Zn_{39.8}) above 9 wt. % metastable solid solution of Zn (Fe Mg) hcp structure was observed.

The Mg in the Fe—Zn—Mg system above may include Mg-containing alloys.

The above also was applied to Fe-based ternary compositions with following compositions (weight %):

Fe_{100-x-y}CaₓZn_{y} wherein 5≤x≤65, 12≤y≤70 and x+y<100.

### 6.0 Conclusion

The results indicated that the metal alloy-containing compositions in accordance with the invention exhibited excellent corrosion behavior. Furthermore, cellular attachment and live/dead assays showed very good attachment of cells, which was superior to controls tested. Thus, the metal alloy-containing compositions in accordance with the invention are deemed suitable for use in fabricating medical implantation devices for applications where controlled degradation and excellent cell compatibility are desired.

### Example 3

### 3.1 Fe—Mg—Mn—X (X=Ca and Zr)

Table 6.0 tabulates the abbreviated notation and chemical composition of Fe—Mg—Mn—X (X=Ca and Zr) alloys.

TABLE 6.0
Summary of chemical formula for Fe—Mg—Mn—X (X = Ca, Zr)
Chemical Formula

| x | Fe₆₀Mg₂₀Mn₂₀₋ₓCaₓ | Fe₆₀Mg₂₀Mn₂₀₋ₓZrₓ |
|---|---|---|
| 0 | Fe₆₀Mg₂₀Mn₂₀ | Fe₆₀Mg₂₀Mn₂₀ |
| 0.5 | — | Fe₆₀Mg₂₀Mn_{19.5}Zr_{0.5} |
| 1 | Fe₆₀Mg₂₀Mn₁₉Ca₁ | — |
| 5 | Fe₆₀Mg₂₀Mn₁₅Ca₅ | Fe₆₀Mg₂₀Mn₁₅Zr₅ |
| 10 | — | Fe₆₀Mg₂₀Mn₁₀Zr₁₀ |
| 15 | — | Fe₆₀Mg₂₀Mn₅Zr₁₅ |
| 20 | — | Fe₆₀Mg₂₀Zr₂₀ |

### 3.2 Fe—Mg—Mn—Ca System

Blended mixtures were prepared from the elemental powder of Fe, Mg, Mn, and Ca and the milled powder was prepared corresponding to the nominal compositions of Fe₆₀Mg₂₀Mn₂₀₋ₓCaₓ powder (x=0, 1, 5). With increasing amounts of Ca of 1 and 5 at. % Ca, the XRD patterns exhibited low intensity sharp peaks from a crystalline phase which were found together with a broad diffraction peak from the corresponding amorphous phase.

Thin layer samples were also prepared by PLD on glass for 30 minutes from the Fe₂₀Mg₂₀Mn₁₅Ca₅ partially amorphous target. This higher Ca containing composition was selected due to the likely higher expected cytocompatibility and better corrosion characteristics that would be expected with the presence of calcium. Glancing angle XRD analysis was performed on this deposited layer. The as-deposited thin layer of the alloy was predominantly amorphous with a small amount of unreacted Fe.

Magnetization (hysteresis) curves of the as milled powder corresponding to the nominal composition Fe₆₀Mg₂₀Mn₂₀₋ₓCaₓ powder (x=0, 1, 5) and stainless-steel alloy, SS316L alloy, showed that compared to SS316L alloy, the as dry milled Fe₆₀Mg₃₅Mn₁₅Ca₅ showed a higher value of M/H slopes with high Mₛ value due to the formation of pure Fe. This is a typical characteristic ferromagnetic behavior. This trend can be interpreted that nonmagnetic phase (x=0 or 1) disappears or transfers to the ferrite phase with increasing Ca content in the alloy.

Based on the potentio-dynamic polarization (PDP) curves for the thin layer of Fe₆₀Mg₂₀Mn₁₅Ca₅, Fe₆₀Mg₂₀Mn₂₀ and pure iron on glass and polished bulk pure iron, taken from representative samples of the multiple samples tested, Table 7 shows a summary of the corrosion potential and corrosion current densities as calculated from extrapolation of the corresponding Tafel plots.

TABLE 7
Corrosion potential (Ecorr) and current density (icorr) values for
Fe₆₀Mg₂₀Mn₁₅Ca₅ layer, both bulk and thin layer of pure iron.
The corrosion current density is significantly higher for
Fe₆₀Mg₂₀Mn₁₅Ca₅ than pure iron in layer samples (P <0.05).

| | Corrosion | Corrosion |
|---|---|---|
| | Potential | Current Density |
| Element | Ecorr (V) | Icorr (μAcm⁻²) |
| Fe₆₀Mg₂₀Mn₁₅Ca₅ thin layer | −0.599 | 18.27 ± 2.11 |
| Fe₆₀Mg₂₀Mn₂₀ amorphous thin layer | −0.621 | 27.07 ± 3.3 |
| Fe₇₀Mg₃₀ amorphous thin layer | −0.642 | 98.76 ± 11.08 |
| Pure Fe (thin layer) | −0.616 | 12.46 ± 2.3 |
| Pure Fe (bulk) | −0.592 | 14.14 ± 4.35 |

The Fe₆₀Mg₂₀Mn₁₅Ca₅ thin layer on glass resulted in an i_{corr} value higher than both Fe thin layer of same thickness of ˜1-3 microns and bulk Fe samples but exhibited E_{corr} values that were more anodic than Fe₆₀Mg₂₀Mn₂₀and Fe₇₀Mg₃₀ amorphous thin layer. It can therefore be inferred from the above results that the Fe₆₀Mg₂₀Mn₁₅Ca₅ alloy shows no improved corrosion behavior compared to the Fe₆₀Mg₂₀Mn₂₀ amorphous thin layer.

Live/Dead staining of MC3T3-E1, hMSCs, HUVECs and NIH3T3 fibroblasts cell on 1 and 3 days of post seeding on the films of Fe₆₀Mg₂₀Mn₁₅Ca₅ were conducted. The fluorescence microscopy images were collected following live/dead assay on the amorphous thin layer and plastic tissue culture surface (control) after 1 day. For MC3T3-E1 and hMSCs cell cultures, the fluorescence microscopy from the live/dead assay showed similar live cells attachment with few dead cells on the surface of Fe₆₀Mg₂₀Mn₁₅Ca₅. After 3 days of culture, the Fe₆₀Mg₂₀Mn₁₅Ca₅ amorphous thin films, which were seeded with MC3T3-E1 and hMSCs still displayed a similar level of living cells attached that is comparable to the tissue culture plastic used as the control, with a higher cell density than day 1. The cells were also more evenly distributed. The HUVECs also showed good cell attachment with few dead cells on the surface of Fe₆₀Mg₂₀Mn₁₅Caₛ compared to the control after day 1. The cells also displayed good cell attachment compared to tissue culture plastic even after 3 days of culture. The cells also seemed to spread with relatively increased cell density compared to 1 day. Whereas, on the other hand, the NIH3T3 fibroblasts cells showed less live cell attachment with considerable dead cells that were largely shrunk compared to the control of tissue culture plastic after 1 day. The result thus indicated inferior cell viability and proliferation on Fe₆₀Mg₂₀Mn₁₅Ca₅ thin layer for NIH3T3 fibroblasts cells. This seemed to arise due to the presence of Mn which appeared to have a high inhibition effect to the NIH3T3 fibroblast cells.

The indirect cytotoxicity results of PLD derived Fe₆₀Mg₂₀Mn₁₅Ca₅ thin layer on glass performed using MC3T3-E1, hMSCs, HUVECs and NIH3T3 fibroblasts cell and the MTT assay were also obtained. The MTT assay conducted with MC3T3-E1 and hMSCs with extract from Fe₆₀Mg₂₀Mn₁₅Ca₅ thin layer displayed low toxicity of the alloy extract, with at least ˜90% cell viability observed for all the culture days. In contrast the results for HUVEC are in the range of 60% for day 1 with slight increment observed reaching ˜65% for day 3 and day 7. Reduction in cell viability by more than 30% is considered to be a good indicator of cytotoxicity. Thus, the alloy film considered here was construed to be non-cytotoxic based on the response seen for MC3T3-E1 and hMSC with HUVEC indicating acceptable cytocompatibility. However, values of cell viability for NIH3T3 fibroblast cell in amorphous Fe₆₀Mg₂₀Mn₁₅Ca₅ thin layer extracts was barely below 50% after 1 day of exposure in agreement with the Live/Dead assay results. The viabilities slightly increased for the subsequent culture days of 3 and 7 but nevertheless still remain below ˜50% compared with that of the negative control after 3- and 7-days of incubation. These results are also in agreement with the Live/Dead assay results discussed above.

3.3 Fe—Mg—Mn—Zr system

The XRD patterns for the as prepared milled powder corresponding to Fe₆₀Mg₂₀Mn₂₀₋ₓZrₓ compositions (x=0.5, 5, 10, 15, 20) were collected and analyzed. The XRD patterns of the powder for all ranges of x in the compositions, showed the typical broad diffraction pattern corresponding to the fully amorphous phase. Further studies were therefore focused on the Fe₆₀Mg₂₀Mn₁₀Zr₁₀ composition with a median value of x representing an optimal amount of Mn and Zr. It was expected that a reduction in the Mn content for this composition offset by larger amount of the more cytocompatible Zr would likely have a more favorable response to the cell viability without compromising the magnetic behavior. The XRD patterns of the starting powder, target, and the deposited films corresponding to this composition all confirmed the amorphous nature.

The hysteresis loop for Fe₆₀Mg₂₀Mn₂₀₋ₓZrₓ (x=0˜20) alloy powder after dry and wet milling was also collected and analyzed. All the samples exhibited almost no hysteresis compared to the stainless-steel alloy, SS316L. All the alloys thus, exhibited non-magnetic properties. An interesting result in this study is the formation of nonmagnetic phase without the presence of Mn in the case of Fe₆₀Mg₂₀₋ₓZrₓ (x=20), which is known to be the austenite stabilizing element. This was an interesting result certainly prompting future work to be conducted to further study the effect of Zr as an alloying element and its influence on the magnetic properties of the Fe—Mg alloy system.

Potentio-dynamic polarization curves (PDP) for the thin layer of Fe₆₀Mg₂₀Mn₁₀Zr₁₀ and pure iron on glass and polished bulk pure iron were prepared. Table 8 gives a summary of the corrosion potential and corrosion current densities as calculated from extrapolation of the Tafel plots. The Fe₆₀Mg₂₀Mn₁₀Zr₁₀ amorphous thin layer on glass yielded an i_{corr} value similar to that of Fe thin layer of similar thickness (˜1-3 micron) and bulk Fe samples as well as E_{corr} values that were more cathodic indicating its lower propensity for undergoing faster degradation compared to bulk Fe and Fe₇₀Mg₃₀ compositions.

TABLE 8
Corrosion potential (Ecorr) and current density (icorr) values for
Fe₆₀Mg₂₀Mn₁₀Zr₁₀ layer, both bulk and thin layer of pure iron.

| | Corrosion | Corrosion |
|---|---|---|
| | Potential | Current Density |
| Element | Ecorr (V) | Icorr (μAcm⁻²) |
| Fe₆₀Mg₂₀Mn₁₅Zr₁₀ thin layer | −0.551 | 13.95 ± 4.01 |
| Fe₆₀Mg₂₀Mn₂₀ amorphous thin layer | −0.621 | 27.07 ± 3.3 |
| Fe₇₀Mg₃₀ amorphous thin layer | −0.642 | 98.76 ± 11.08 |
| Pure Fe (thin layer) | −0.616 | 12.46 ± 2.3 |
| Pure Fe (bulk) | −0.592 | 14.14 ± 4.35 |

Live/Dead staining of MC3T3-E1, hMSCs, HUVECs and NIH3T3 after 1 and 3 days of post seeding on the Fe₆₀Mg₂₀Mn₁₀Zr₁₀ amorphous thin layer and plastic tissue culture surface (control) were also conducted. After 1 day, for MC3T3-E1, hMSCs and HUVECs cultures, fluorescence microscopy images collected from the live/dead assay showed similar live cells attachment with few dead cells on the surface of Fe₆₀Mg₂₀Mn₁₀Zr₁₀ films compared to the control. After 3 days of culture, Fe₆₀Mg₂₀Mn₁₀Zr₁₀ amorphous thin films, which were seeded with all of the MC3T3-E1, hMSCs and HUVECs cell lines, still displayed a similar level of living cell attachment, comparable to the tissue culture plastic used as the control, with a higher cell density than day 1. The cells were also more evenly distributed. The HUVECs cell lines also showed good cell attachment with few dead cells on the surface of Fe₆₀Mg₂₀Mn₁₅Ca₅ compared to the control after day 1. The live/dead staining indicated good cell viability and proliferation on F₆₀Mg₂₀Mn₁₀Zr₁₀ amorphous thin layer. Whereas, on the other hand, the NIH3T3 fibroblasts cells showed less live cell attachment with dead cells and shrunken morphology for the cells compared to the control after 1 day with continued indication of reduced number of live cells attached to the thin film surface compared to the tissue culture plastic at day 3. The live/dead staining results thus indicated relatively good cell viability and proliferation for MC3T3-E1, hMSCs and HUVECs with some likely cytotoxicity for NIH3T3 fibroblasts cells as was observed for the Fe—Mg—Mn—Ca systems discussed earlier.

To further corroborate the live/dead assay results, indirect MTT assay studies were also conducted. The cell viabilities of MC3TC-E1, hMSCs, HUVECs and NIH3T3 were expressed as a percentage of the viability of cell cultured in the negative control after 1, 3, and 7days incubation in 100% extraction mediums after 72 hours using the MTT assay. These quantitative results of indirect cell viability indicated good cell viability and proliferation on Fe₆₀Mg₂₀Mn₁₀Zr₁₀ amorphous thin layers. For 1, 3 and 7 days of culture, cell viabilities of MC3TC-E1 indicated slight variations although similar to that of pure Fe. There is also no significant difference (P>0.05) among them although there was consistently lower cell viability compared to the negative control. The values of cell viability for hMSCs and HUVECs on 100% extracts collected over amorphous Fe₆₀Mg₂₀Mn₁₀Zr₁₀ showed 70% after 1day of exposure. The viabilities however, slightly increased indicating values ˜75% compared with that of negative control after 3- and 7-days' of incubation for HUVECs and hMSCs while exhibiting ˜80% or higher viabilities for HUVECs following day 3 and 7. For NIH3T3 cell however, the cell viability was about 60% after 1day incubation and remained in the same range following day 3 and 7 incubation. The MC3T3-E1 cells on the other hand, displayed almost ˜80% cell viability after 1 day of exposure increasing to above ˜90% of the negative control without extract after 7 days of incubation. These results therefore indicated that the amorphous layers of Fe₆₀Mg₂₀Mn₁₀Zr₁₀ showed good cell viability for all cells except for the NIH3T3 fibroblasts cell akin to what was observed for the Fe—Mg—Mn—Ca alloy system discussed earlier.

### 3.4 Conclusions

Results of the studies discussed above indicate that addition of Ca or Zr to Fe—Mg and Fe—Mg—Mn systems can generate amorphous powders following HEMA as well as amorphous films by PLD. The in vitro effects of the alloying elements Ca or Zr on Fe—Mg and Fe—Mg—Mn alloys were also investigated. The results exhibited good effects of addition of alloying elements to form new biodegradable Fe-based alloys for use as medical device applications. The systems were studied in terms of corrosion, magnetic properties as well as cytocompatibility.

The results indicate that by addition of Ca as an alloying element to Fe₇₀Mg₃₀ amorphous alloy results in the successful generation of Fe₇₀Mg₂₂Ca₈ thin layer by PLD from HEMA derived powders compacted to form targets of the same crystal structure and chemical composition. The powders and the films exhibited partial amorphous structures with unreacted pure Fe. The in vitro corrosion results indicated corrosion current density value was approximately 2-fold higher than pure Fe but slower compared to that of Fe₇₀Mg30 amorphous thin layer. The direct and indirect cytotoxicity results also indicated that Fe₇₀Mg₂₂Ca₈ thin layer exhibits no cytotoxicity to MC3T3-E1, hMSCs, HUVECs and NIH3T3 cell lines.

By adding the transition element Zr to Fe₇₀Mg₃₀ amorphous alloy, the amorphous composition of Fe₇₀Mg₂₈Zr₂ thin layer was deposited by PLD. For corrosion behavior, it was determined that the composition exhibited rapid corrosion compared to pure Fe although slower compared to the Fe₇₀Mg₃₀ amorphous thin layer. The MTT assay conducted with all the cell lines with 100% extract from Fe₇₀Mg₂₈Zr₂ thin layer displayed low toxicity of the alloy extract, with at least ˜80% cell viability observed for all the three cell culture conditions of day 1, day 3 and day 7. Thus, the alloys may be considered non-cytotoxic.

For alloying element Ca on Fe₆₀Mg₂₀Mn₂₀ amorphous alloy, Fe₆₀Mg₃₅Mn₁₅Ca₅ thin layer was successfully synthesized by PLD from HEMA derived powder compacted targets of the same crystal structure and chemical composition. The films exhibited partial amorphous phase and, also, additionally, exhibit ferromagnetic property in contrast to Fe₆₀Mg₂₀Mn₂₀ amorphous alloy. Fe₆₀Mg₃₅Mn₁₅Ca₅ thin layer still exhibited rapid corrosion compared to pure Fe but is lower than Fe₆₀Mg₂₀Mn₂₀ amorphous thin layer. The films showed good cell viability results for all cells except NIH3T3 fibroblasts cell.

Further, Fe₆₀Mg₂₀Mn₁₀Zr₁₀ amorphous thin layer was successfully synthesized by PLD from HEMA derived compacted powders comprising the same crystal structure and chemical composition. All the powders exhibited nonmagnetic characteristics although the corrosion behavior appeared to be similar to pure Fe. The direct and indirect cytotoxicity results indicated the Fe₆₀Mg₂₀Mn₁₀Zr₁₀ amorphous thin layer may be considered non-cytotoxic for MC3T3-E1, hMSCs and HUVECs except for the NIH3T3 cell lines.

The results discussed above show that addition of Mn is critical to induce antiferromagnetic properties to the amorphous Fe₇₀Mg₃₀ system. However, cell viability appeared to be variable due to the toxicity associated with Mn particularly, with NIH3T3 and HUVEC lines. Addition of 20 at % Zr with the absence of Mn, however, transferred anti-ferromagnetic characteristics to the amorphous phase of Fe₇₀Mg₃₀ showing the potential of this system serving as a viable system.

1. A biodegradable iron-based alloy, comprising:
magnesium, manganese and calcium; and
a remainder of iron.

2. The alloy of claim 1, wherein the iron constitutes from about 10.0 weight percent to about 60.0 weight percent, the magnesium constitutes from greater than zero weight percent to about 20.0 weight percent, the manganese constitutes from about 5.0 weight percent to about 19.0 weight percent, and the calcium constitutes from greater than zero weight percent to about 1.0 weight percent, based on total weight of the composition.

3. The alloy of claim 1, wherein the iron constitutes from about 10.0 weight percent to about 60.0 weight percent, the magnesium constitutes from greater than zero weight percent to about 20.0 weight percent, the manganese constitutes from about 5.0 weight percent to about 15.0 weight percent, and the calcium constitutes from greater than zero weight percent to about 5.0 weight percent, based on total weight of the composition.

4. The alloy of claim 1, wherein the iron constitutes from about 10.0 weight percent to about 60.0 weight percent, the magnesium constitutes from greater than zero weight percent to about 35.0 weight percent, the manganese constitutes from about 5.0 weight percent to about 15.0 weight percent, and the calcium constitutes from greater than zero weight percent to about 5.0 weight percent, based on total weight of the composition.

5. A biodegradable iron-based alloy, comprising:
magnesium, manganese and zirconium; and
a remainder of iron.

6. The alloy of claim 5, wherein the iron constitutes from about 10.0 weight percent to about 60.0 weight percent, the magnesium constitutes from greater than zero weight percent to about 20.0 weight percent, the manganese constitutes from about 5.0 weight percent to about 19.5 weight percent, and the zirconium constitutes from greater than zero weight percent to about 0.5 weight percent, based on total weight of the composition.

7. The alloy of claim 5, wherein the iron constitutes from about 10.0 weight percent to about 60.0 weight percent, the magnesium constitutes from greater than zero weight percent to about 20.0 weight percent, the manganese constitutes from about 5.0 weight percent to about 15.0 weight percent, and the zirconium constitutes from greater than zero weight percent to about 5.0 weight percent, based on total weight of the composition.

8. The alloy of claim 5, wherein the iron constitutes from about 10.0 weight percent to about 60.0 weight percent, the magnesium constitutes from greater than zero weight percent to about 20.0 weight percent, the manganese constitutes from about 5.0 weight percent to about 10.0 weight percent, and the zirconium constitutes from greater than zero weight percent to about 10.0 weight percent, based on total weight of the composition.

9. The alloy of claim 5, wherein the iron constitutes from about 10.0 weight percent to about 60.0 weight percent, the magnesium constitutes from greater than zero weight percent to about 20.0 weight percent, the manganese constitutes from greater than zero to about 5.0 weight percent, and the zirconium constitutes from greater than zero weight percent to about 15.0 weight percent, based on total weight of the composition.

10. A method of preparing a biodegradable iron-based alloy, comprising:
charging in a high energy mill (i) magnesium, manganese, calcium, and a remainder of iron, or (ii) magnesium, zirconium, calcium, and a remainder of iron, or (iii) magnesium, zinc, calcium, and a remainder of iron, or (iv) magnesium, manganese, zirconium, and a remainder of iron; and
conducting high energy milling of one of the (i), (ii), (iii) or (iv).

11. The method of claim 10, wherein the high energy milling is conducted in dry conditions followed by high energy milling conducted in wet conditions.

12. The method of claim 10, wherein a material resulting from the high energy milling is subjected to a melting and casting process to form an iron-based alloy cast into an alloy.

13. A biodegradable medical device comprising the iron-based alloy of claim 1.

14. A biodegradable medical device comprising the iron-based alloy of claim 5.

15. The device of claim 13, wherein the said device is selected from the group consisting of plate, mesh, staple, screw, pin, tack, rod, suture anchor, tubular mesh, coil, x-ray marker, catheter, pipe, shield, bolt, clip, plug, dental and craniofacial implant, graft device, endoprosthesis, bone-fracture healing device, bone void filler device, bone replacement device, joint replacement device, tissue regeneration device, cardiovascular stent, tracheal stent, nerve guide, surgical implant, surgical wire, Kirschner wire, cerclage wire, and combinations thereof.

16. The device of claim 13, wherein said device is an implantable device.

17. The device of claim 16, wherein the implantable device is selected from the group consisting of an orthopedic device, a craniofacial device, tracheal device, and a cardiovascular device.

18. The device of claim 14, wherein said device is selected from the group consisting of plate, mesh, staple, screw, pin, tack, rod, suture anchor, tubular mesh, coil, x-ray marker, catheter, pipe, shield, bolt, clip, plug, dental and craniofacial implant, graft device, endoprosthesis, bone-fracture healing device, bone void filler device, bone replacement device, joint replacement device, tissue regeneration device, cardiovascular stent, tracheal stent, nerve guide, surgical implant, surgical wire, Kirschner wire, cerclage wire, and combinations thereof.

19. The device of claim 14, wherein said device is an implantable device.

20. The device of claim 19, wherein the implantable device is selected from the group consisting of an orthopedic device. a craniofacial device, a tracheal device, and a cardiovascular device.
